(19) United States
(12) Patent Application Publication
(10) Pub. No.: US 2011/0100368 A1
(43) Pub. Date: May 5, 2011

(54) Face mask for endoscopy

(76) Inventor: Lisa Carole Taylor-Kennedy, Dallas County, TX (US)

(21) Appl. No.: 12/590,193

(22) Filed: Nov. 4, 2009

(51) Int. Cl. A61M 16/06; A61B 1/00

(52) U.S. Cl. 128/205.25; 600/102

(57) ABSTRACT

A face mask assembly according to the present invention, includes a mask body. The inside of the mask body is of a size and shape to substantially conformally fit over the oral and nasal region of a patient's face. The mask body forms a receptacle and an opening above the receptacle. The face mask assembly also includes an inlet connection device connected to the receptacle and a hose connected to the inlet connection device. The hose is in open communication with the inside of the mask body to supply gas, normally oxygen. The opening formed above the receptacle is of sufficient size and shape to allow use of a medical scope such as an endoscope or a bronchoscope or a transesophogeal echocardiography scope or similar device. A closure flap substantially closes the opening when the opening is not being used for a medical scope. A port, also connected to the receptacle, is used to sample end of tidal carbon dioxide.

### CROSS-REFERENCE TO RELATED APPLICATIONS

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

1. Field of the Invention

The field of the invention is face masks generally, and more specificaly, medical face masks.

2. Description of Related Art

Supplemental oxygen is frequently required during endoscopic procedures including upper gastrointestinal endoscopy, bronchoscopy and transesophageal echocardiography. Supplemental oxygen is often given by means of a nasal cannula because oxygen given by means of a face mask would interfere with the endoscopic procedures.

Various attempts have been made to solve the problem of supplying oxygen for endoscopic procedures. U.S. Pat. No. 4,328,797 shows a face mask for a naso-gastric intubation procedure, but the entry point is on the side of the mask, at a right angle to the desired entry point on the patient, and also several inches removed.

U.S. Pat. No. 4,580,556, issued to Kondur, shows an adaptor for supplying anesthetic during endotracheal intubation. The adaptor, however, has entry through the anesthetic supply connection, possibly interfering with the supply, but also making entry of the endotracheal tube and the fiberoptic laryngoscope at a difficult angle from several inches away from the patient.

Published United States Patent Application No. US 2003/0024533 shows the use of a multi-port face mask. Once again, the nasal port is at a difficult angle and is extended so that it is several inches removed from the desired entry area of the patient.

### BRIEF SUMMARY OF THE INVENTION

A face mask assembly according to the present invention, includes a mask body. The inside of the mask body is of a size and shape to substantially conformally fit over the oral and nasal region of a patient's face. The mask body forms a receptacle and an opening above the receptacle. The face mask assembly also includes an inlet connection device connected to the receptacle and a hose connected to the inlet connection device. The hose is in open communication with the inside of the mask body. The opening formed above the receptacle is of sufficient size and shape to allow use of a medical scope such as an endoscope or a bronchoscope or a transesophogeal echocardiography scope or similar device.

In a preferred form, a face mask assembly according to the present invention, further includes a closure flap to substantially close the opening when the opening is not being used for a medical scope. In another preferred form, the hose is used for supplying oxygen to the patient.

Another preferred form of the face mask assembly includes a port also connected to the receptacle, to sample end of tidal carbon dioxide.

These and other objects, advantages and features of this invention will be apparent from the following description taken with reference to the accompanying drawing, wherein is shown a preferred embodiment of the invention.

### BRIEF DESCRIPTION OF THE SEVERAL VIEWS OF THE DRAWING

FIG. 1 is a front elevation view of a patient wearing a face mask assembly according to the present invention; and

FIG. 2 is a right side elevation view of the face mask assembly of FIG. 1.

### DETAILED DESCRIPTION OF THE INVENTION

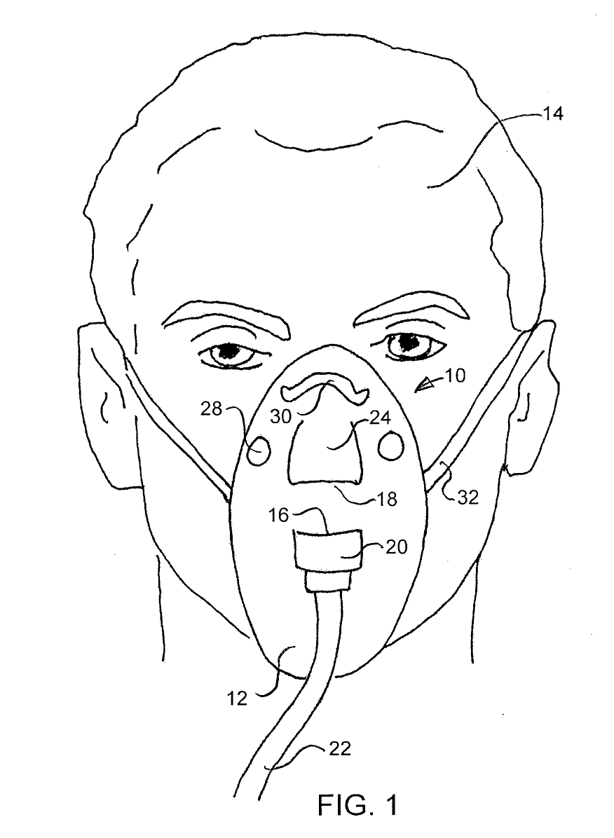

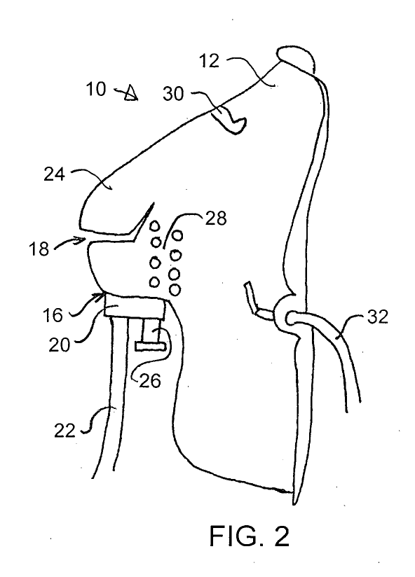

Referring now to the drawing, and in particular to FIG. 1 and FIG. 2, a face mask assembly according to the present invention is referred to generally by reference numeral 10. Face mask assembly 10 includes a mask body 12. The inside of the mask body, the side facing patient 14, is of a size and shape to substantially conformally fit over the oral and nasal region of the patient's face, which is to say fit along the edge of the mask, giving ample room inside the mask for patient comfort. Mask body 12 forms a receptacle 16 and an opening 18 above the receptacle. Face mask assembly 10 also includes a relatively rigid inlet connection device 20 connected to receptacle 16 and a hose 22 connected to the inlet connection device. The hose is in open communication with the inside of the mask body. Opening 18 formed above receptacle 16 is of sufficient size and shape to allow use of a medical scope such as an endoscope or a bronchoscope or a transesophogeal echocardiography scope or similar device.

In a preferred form, face mask assembly 10 according to the present invention, further includes a closure flap 24 to substantially close the opening when the opening is not being used for a medical scope. Closure flap 24 can be integral to face mask body 12 as shown or it can be separate from the face mask body, attached by a hinge or pin. Closure flap 12 can also be free with a tendency to close as illustrated or it can have a latch or snap closure. In another preferred form, hose 22 is used for supplying oxygen to patient 14.

Another preferred form of the face mask assembly includes a port 26, not visible in FIG. 1, also connected to inlet connection device 20, to sample end of tidal carbon dioxide.

Thus, oxygen can be fed through the hose 22 into the mask body 12 through the connector 20 for increasing and controlling the oxygen intake of patient 14. In order to allow the exhaled breath of the patient to be exhausted from the mask, one or more apertures 28 may be provided in either or both sides of the mask. One or more apertures 28 may be sealed for outflow only.

If desired, the upper portion of the mask may be provided with a pliable nose piece 30 which may be bent to generally conform to the nose of the patient. The nose piece assists in holding the mask in place and keeping it sealed against the patient's face. Similarly, the mask may be provided with a band, strap or earpiece 32 in order to hold the mask to the patient's face.

It can now be seen that a face mask according to the present invention overcomes many of the problems of the prior art. The opening is directly above the patient's nose and is close enough and large enough to easily insert an endoscopic device. At the same time, the mask automatically seals the mask when the opening is not being used procedurally.

From the foregoing it will be seen that this invention is well adapted to attain all of the ends and objectives hereinabove set forth, together with other advantages which are inherent to the apparatus.

It will be understood that certain features and subcombinations are of utility and may be employed without reference to other features and subcombinations. This is contemplated by and is within the scope of the claims.

As many possible embodiments may be made of the invention without departing from the scope thereof, it is to be understood that all matter herein set forth or shown in the figures of the accompanying drawings is to be interpreted as illustrative and not in a limiting sense.

1. A face mask assembly comprising, in combination:
a mask body, the inside of the mask body being of a size and shape to substantially conformally fit over the oral and nasal region of a patient's face, the mask body forming a receptacle and an opening above the receptacle;
an inlet connection device connected to the receptacle;
a hose connected to the inlet connection device, wherein the hose is in open communication with the inside of the mask body;
wherein the opening formed above the receptacle is of sufficient size and shape to allow use of a medical scope such as an endoscope or a bronchoscope or a transesophogeal echocardiography scope or similar device.

2. A face mask assembly according to claim 1, further including a closure flap to substantially close the opening when the opening is not being used for a medical scope.

3. A face mask assembly according to claim 2, wherein the hose is used for supplying oxygen to the patient.

4. A face mask assembly according to claim 3, further including a port also connected to the inlet connection device, to sample end of tidal carbon dioxide.
